(19) United States
(12) Patent Application Publication
(10) Pub. No.: US 2026/0249074 A1
(43) Pub. Date: Aug. 27, 2026

(54) METHOD AND APPARATUS FOR IMPROVING WEARABLE ELECTRODE DURABILITY

(71) Applicant: AVATION MEDICAL, INC., Columbus, OH (US)

(72) Inventors: Austin MORGAN, Westerville, OH (US); Manish VAISHYA, Valencia, CA (US)

(21) Appl. No.: 19/545,430

(22) Filed: Feb. 20, 2026

Related U.S. Application Data
(60) Provisional application No. 63/762,753, filed on Feb. 25, 2025.

(51) Int. Cl. A61N 1/04; B32B 9/04; B32B 27/08; B32B 27/40; B41M 1/12; B41M 1/30; B41M 7/00; H01B 5/14; H01B 13/00

(52) U.S. Cl. CPC A61N 1/0456; A61N 1/0484; B32B 9/045; B32B 27/08; B32B 27/40; B41M 1/12; B41M 1/30; B41M 7/0027; H01B 5/14; H01B 13/0036; B32B 2255/10; B32B 2307/202; B32B 2457/00

(57) ABSTRACT

An electrode for applying transcutaneous electrical stimulation includes a flexible substrate, an electrically conductive contact pad printed on the substrate with conductive ink, and a reinforcing layer applied onto the substrate and covering a periphery of the contact pad. The reinforcing layer is configured to reinforce the periphery of the contact pad to inhibit material failures of the conductive ink along the periphery. The reinforcing layer is also configured to maintain electrical continuity along the periphery of the contact pad in order to provide electrical continuity across portions of the contact pad within the periphery that are left exposed by the reinforcing layer.

### RELATED APPLICATION

This application claims the benefit of U.S. Provisional Application Ser. No. 63/762,753, filed on Feb. 25, 2025, the disclosure of which is hereby incorporated by reference in its entirety.

### TECHNICAL FIELD

This application relates to medical devices in the field of electrical neurostimulation. In one implementation, the application relates to a method and apparatus for improving neurostimulator systems that employ the use of wearable electrodes to apply transcutaneous electrical neurostimulation targeting peripheral nerves.

### BACKGROUND

There are many known technologies that use electrical stimulation of peripheral nerves. Implantable stimulation technologies require surgical implantation of stimulation leads, with a pulse generator that is either surgically implanted or connected externally to wire leads. Percutaneous stimulation technologies are less invasive, but still require the stimulation electrodes to pierce the skin. While these technologies can be effective in treating certain conditions, they are less desirable due to their invasiveness and because they can require the continued or routine attention of specialists, requiring doctor's office visits, phone calls, etc. Transcutaneous neurostimulation systems employ the use of surface electrodes that avoid the issues described above regarding implantable or percutaneous stimulation techniques.

Wearable transcutaneous stimulation and/or sensing systems can include electrodes supported on a wearable garment, strap, brace, etc., where the electrodes are formed on the garment using printed conductive inks on flexible polymer substrates. During use, a wearable may undergo repeated bending, stretching, torsion, compression, and shear caused by normal motion of the subject (e.g., walking, running, grasping, or other activities). Cyclic deformation can produce microcracks, fissures, tears, or other discontinuities in the printed conductive material and/or at interfaces between layers, leading to changes in electrode impedance and resistance, spatially non-uniform current density, increased stimulation voltage requirements, degraded sensing fidelity (e.g., reduced signal-to-noise ratio for sensing electrodes), increased motion artifacts, and increased session-to-session variability.

Accordingly, there is a need for wearable electrode constructions and associated manufacturing methods that improve durability of printed conductive electrodes under cyclic deformation while maintaining electrical continuity and stable electrical characteristics suitable for both stimulation and sensing applications.

### SUMMARY

A system for applying transcutaneous electrical neurostimulation includes an electronic stimulator that controls the delivery of a transcutaneous electrical neurostimulation signal via wearable surface stimulation electrodes applied in contact with the subject's skin. The stimulator includes a controller that modulates the electrical neurostimulation signal to stimulate the target nerve through the skin via the stimulation electrodes according to a prescribed treatment regimen. The system also includes recording electrodes configured to detect electromyographical (EMG) responses from muscles that are activated in response to stimulation of the target nerve. The controller can be configured to control the application of electrical neurostimulation applied via the stimulation electrodes in response to the EMG responses detected via the recording electrodes.

In certain implementations, the wearable electrodes are configured to maintain one or more electrical characteristics within a predetermined range over repeated use. These electrical characteristics can include, for example, contact pad resistance, contact pad impedance, current density distribution, stimulation threshold, recorded EMG amplitude, recorded EMG signal-to-noise ratio, and motion artifact level.

The stimulation and recording electrodes are wearable surface electrodes. The surface electrodes include a substrate upon which a conductive ink is printed to form one or more electrode pads, as well as traces configured to conduct electrical signals between the pads and the controller. An adhesive applied to the surface of the electrodes adheres the electrodes to the skin surface with the electrode pads contacting the skin surface.

Due to differences in the elasticity of the substrate and the conductive ink, substrate stretching can cause material failures, such as fissures, cracks, or tears in contact pads of the electrodes, which can affect their electrical properties and can negatively affect their performance. The wearable surface electrodes include a peripheral reinforcing element configured to prevent stretching of the substrate due to the subject's moving during use in order to prevent these material failures. Additionally, the reinforcing element can include a peripheral conductive element configured to engage the peripheries of the contact pads and to provide electrical conductivity across any material failures that appear in the peripheries.

Accordingly, an electrode for applying transcutaneous electrical stimulation includes a flexible substrate, an electrically conductive contact pad printed on the substrate with conductive ink, and a reinforcing layer applied onto the substrate and covering a periphery of the contact pad. The reinforcing layer is configured to reinforce the periphery of the contact pad to inhibit material failures of the conductive ink along the periphery. The reinforcing layer is also configured to maintain electrical continuity along the periphery of the contact pad in order to provide electrical continuity across portions of the contact pad within the periphery that are left exposed by the reinforcing layer.

According to one aspect, the flexible substrate can include a first polymer sheet onto which the contact pad is printed, and the reinforcing layer can include a second polymer sheet laminated onto the flexible substrate.

According to another aspect, the reinforcing layer can be applied by printing, coating, spraying, transfer, lamination, heat bonding, ultrasonic welding, adhesive bonding, or combinations thereof. The reinforcing layer can be formed as a patterned layer (e.g., ring, frame, lattice, ribs, islands) configured to reduce strain while preserving overall flexibility of the electrode.

According to one aspect, the substrate and reinforcing layer can be formed from thermoplastic polyurethane (TPU) sheets. Alternatively, the substrate and/or reinforcing layer can include one or more elastomeric or polymeric materials including, for example, polyurethane, TPU blends, silicone, silicone-polyurethane hybrids, polyolefins, polyesters, polyimides, or multilayer laminates.

According to one aspect, the reinforcing layer can include a layer of conductive material configured to overlie the periphery of the contact pad.

According to one aspect, the layer of conductive material can be configured to contact the periphery of the contact pad to provide electrical conductivity across material failures of the contact pad. In certain embodiments, this peripheral conductive ring is electrically coupled to the contact pad at multiple locations around the periphery, thereby providing redundant current paths around cracks and maintaining equipotential behavior across the pad.

According to one aspect, the electrode can also include an encapsulant ink layer that covers the contact pad.

According to one aspect, the encapsulant ink layer can fully cover contact pad.

According to one aspect, the encapsulant ink layer can include a perforate layer that allows direct current transmission via perforations that expose portions of the contact pad.

According to one aspect, the encapsulant ink layer can partially cover contact pad and leaves exposed portions of the contact pad configured for skin contact.

In certain embodiments, the encapsulant ink layer is configured to reduce abrasion and environmental exposure (e.g., moisture, oils, sweat) and to reduce crack initiation at the surface of the printed conductive material.

According to one aspect, the electrode can also include a hydrogel pad configured to overlie the contact pad.

According to one aspect, an apparatus for applying transcutaneous electrical stimulation can include the electrode mounted on a wearable structure configured to be worn by a subject. The apparatus can include a controller configured to control the operation of the electrode to apply electrical stimulation energy to the subject.

According to one aspect, the at least one electrode can include an electrode array comprising a plurality of the electrodes. The controller can be configured to selectively activate subsets of electrodes for stimulation and/or recording to perform current steering and/or nerve localization.

According to one aspect, a neurostimulation system can include the apparatus for applying transcutaneous electrical stimulation. The controller can be configured to deliver stimulation via at least one stimulation electrode of the plurality of electrodes and to record electromyogram (EMG) responses via at least one recording electrode of the plurality of electrodes. The reinforced periphery of the contact pads of the at least one stimulation electrode can help maintain electrical continuity along the periphery of the contact pad in order to provide electrical continuity across portions of the contact pad within the periphery so that stimulation can be applied evenly across the contact pad despite the formation of discontinuities in the contact pad. The reinforced periphery of the contact pads of the at least one recording electrode can help maintain electrical continuity along the periphery of the contact pad in order to provide electrical continuity across portions of the contact pad within the periphery so that the EMG responses can be recorded with reduced drift despite the formation of discontinuities in the contact pad.

According to one aspect, a method of manufacturing an electrode for transcutaneous electrical stimulation includes printing an electrically conductive contact pad on a flexible polymer substrate using a conductive ink. The method also includes applying a reinforcing layer over a peripheral region of the contact pad while leaving a central region exposed. The method further includes coupling the electrode to a wearable structure. The reinforcing layer reduces strain in the peripheral region during cyclic deformation to inhibit crack formation in the conductive ink.

According to one aspect of the method, applying the reinforcing layer can include laminating a polymer film onto the substrate.

According to one aspect, the method can also include applying a conductive peripheral layer that overlies and electrically couples to the peripheral region to provide redundant conductive paths across cracks.

According to one aspect, an electrode for transcutaneous electrical stimulation includes a flexible substrate, a printed conductive contact pad, and a reinforcing structure coupled to a peripheral region of the contact pad. The electrode is configured such that an electrical resistance and/or impedance of the contact pad remains within a predetermined range after repeated bending and/or stretching cycles representative of wear.

According to one aspect, the reinforcing structure can include a peripheral frame that remains electrically conductive around the contact pad to maintain electrical continuity between discontinuous regions of the contact pad.

According to one aspect, directional anisotropy of resistance across the contact pad is reduced relative to an otherwise identical electrode with discontinuous regions but lacking the reinforcing structure.

### DRAWINGS

The foregoing and other features of the present disclosure will become apparent to one skilled in the art to which the present disclosure relates upon consideration of the following description with reference to the accompanying drawings, in which:

FIG. 1 is a schematic representation of a neurostimulation system according to an example configuration.

FIGS. 2A and 2B illustrate the application of surface electrodes of the neurostimulation system applied via a garment that positions the surface electrodes against skin surfaces of a subject.

FIG. 3 is a plan view illustrating the structure of a surface electrode according to an example configuration.

FIG. 4 is a magnified view of a portion of an electrode exhibiting material failures brought on by continued use.

FIGS. 5A-5B are sectional views of the surface electrode of FIG. 3 taken generally along line 5-5 in FIG. 3, according to an example configuration.

FIG. 5C is a top view of certain portions of the surface electrode of FIGS. 5A and 5B.

FIGS. 6A-6B are sectional views of the surface electrode of FIG. 3 taken generally along line 6-6 in FIG. 3, according to another example configuration.

FIG. 6C is a top view of certain portions of the surface electrode of FIGS. 6A and 6B.

FIGS. 7A and 7B are sectional views of the wearable surface electrode according to additional example configurations.

### DESCRIPTION

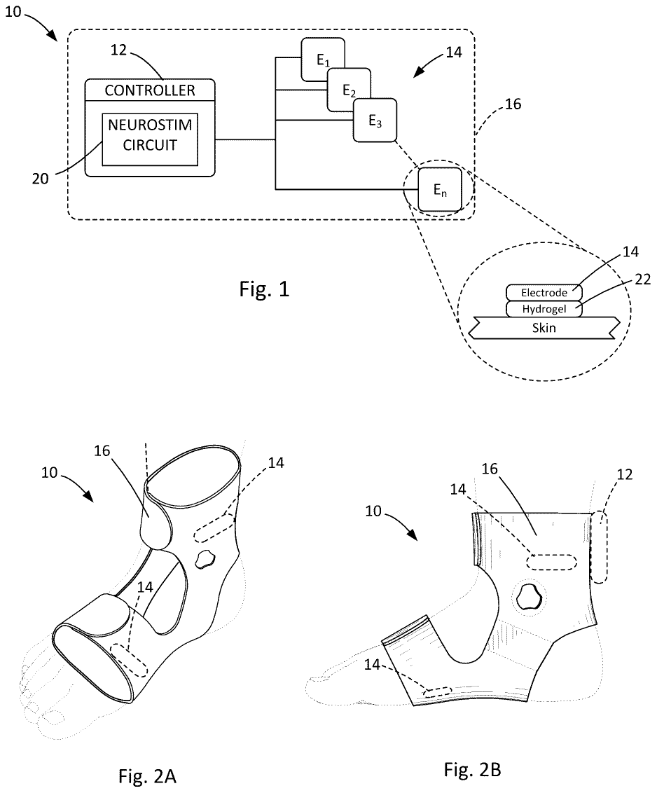

FIG. 1 illustrates an example system 10 for delivering transcutaneous neurostimulation to a subject. The neurostimulation system 10 includes an apparatus in the form of a controller 12 (e.g., a microcontroller) and one or more electrodes 14 (E1, E2, E3, . . . En) configured to be positioned on a skin surface. The positioning of the electrodes 14 on the skin surface can be achieved in a variety of manners. For example, the electrodes 14 can be manually positioned on the skin surface, such as by placing stick-on disposable electrodes directly on the skin. In other implementations, the system 10 can include a wearable 16, such as a strap, brace, sock, sleeve, wrap, or garment, upon which the electrodes 14 and/or the controller 12 can be mounted. In this configuration, the electrodes can be positioned in contact with the skin surface when the wearable 16 is placed on the subject.

As shown in the detail of FIG. 1, regardless of the implementation, a hydrogel pad 22 can be positioned between the electrode 14 and the patient's skin. The hydrogel pad 22 can provide an electrical interface between the electrode 14 and the skin. The hydrogel pad 22 can conform to the skin and provide a reliable physical contact with the patient through which the electrical connection can be established.

The system 10 is configured to apply electrical stimulation signals to one or more nerves of the subject through the skin according to a prescribed neurostimulation method. While these methods can vary widely, they all entail varying or modulating the applied electrical neurostimulation signal, a process that can be referred to as neuromodulation. To do so, a neurostimulation circuit 20 embedded in the controller 12 includes a constant current source that is controlled to produce the neuromodulation signal. The neurostimulation circuit 20 is capable of high voltage biphasic output applied across a load which, in the neurostimulation setting, includes the stimulated tissue.

The neurostimulation system 10 can be configured to implement the electrodes 14 as stick-on electrodes where an adhesive is used to adhere the electrode directly to the skin. In this configuration, if hydrogel pads 22 are used, the adhesive can completely or partially surround the hydrogel pads so that the electrodes 14 are pressed against the hydrogel pads, which presses the hydrogel pads against the skin when the electrode is adhered to the skin surface. In this implementation, the controller 12 is connected to the electrodes via wires 18.

Alternatively, the neurostimulation system 10 can be implemented in a wearable 16, such as a garment, sock, sleeve, brace, strap, etc. The wearable includes the components of the apparatus, e.g., the controller 12 and electrodes 14 with any hydrogel pads 22 being affixed thereto, e.g. via an adhesive. Advantageously, in this implementation, the garment itself secures the electrodes 14 and hydrogel pads 22 against the skin, thus avoiding the need for skin surface adhesives.

As another advantage, because the controller 12 can be supported by the wearable 16, the wiring 18 between the controller and the electrodes 14 can also be supported by the wearable structure. For example, the wiring 18 can be formed by traces printed directly onto the wearable, by wires supported on or in the wearable, or by a combination thereof. Implementation of the neurostimulation system 10 in a wearable is further advantageous in that it allows the subject to use the system at a time and place that is convenient. The subject may choose to use the device while they are at work or at home, or while walking, relaxing, or sleeping, as long as certain environments and/or activities (e.g., wet environments/activities) are avoided. Since there are no implantable or percutaneous components, the risk of infection, battery fault burns, and transcutaneous power transfer discomfort and/or bleeding, are greatly reduced or eliminated.

The wearable 16 includes the electrodes 14/hydrogel pads 22 arranged in a predetermined pattern or array, and that engage the subject's skin at desired locations when the wearable is worn. The electrodes can include stimulating electrodes and recording electrodes, which the wearable can position at the same location or at different locations on the subject's skin. In fact, the identities of individual electrodes, i.e., stimulating or recording, can change depending on the application/treatment for which the system is being used. The stimulating electrodes apply the transcutaneous electrical stimulation to the subject's skin, and the recording electrodes record the electromyogram (EMG) responses elicited by the stimulation.

The wearable 16 positions the electrodes 14 at a target anatomical region and constrains motion of the electrodes relative to skin. Improved durability of the electrodes 14 can reduce changes in effective electrode area and contact impedance over time, thus improving the repeatability and reliability of both stimulation delivery and EMG sensing. In certain embodiments, improved electrode durability maintains sensing fidelity over time such that closed-loop control based on EMG (and/or MMG) remains stable across multiple sessions without requiring manual electrode replacement or repositioning.

The controller 12 is electrically connected to the electrodes 14 and is operable to control electrical stimulation applied by the stimulating electrodes and to control the recording of EMG responses by the recording electrodes. The controller 12 can execute closed-loop control algorithms, which adjust stimulation patterns, periodically or constantly, based on the elicited EMG response from the recruited nerves as feedback. Alternatively, the system 10 can implement open-loop control where stimulation is applied without feedback.

Closed-loop control can eliminate the need for programming sessions commonly required for neurostimulation systems. The day-to-day variability that arises due to electrode placement and skin impedance necessitates these sessions to make sure that the electrodes are positioned to provide adequate stimulation treatment. With the present system, instead of physically adjusting the electrode positions on the subject in order to find the arrangement that produces the desired response, the system itself can select which electrodes to use, and can adjust the number and pattern of electrodes until an acceptable response (EMG and/or MMG) is achieved.

Once the appropriate electrodes pattern is identified, the order, intensity, timing, etc. of the stimulation can be further tuned or adjusted to optimize the EMG and/or MMG response. The system can tailor the electrical stimulation applied by each individually controllable electrode in the array so that the stimulation characteristics of each electrode (e.g., frequency, amplitude, pattern, duration, etc.) is configured to deliver the desired stimulation effect. This tailoring can be implemented automatically through the algorithm, which incrementally adjusts these characteristics, monitoring the EMG and/or MMG response at each increment until optimal settings are identified. Stimulation therapy can then be applied with these settings, according to the algorithm, which can be dictated by the requirements of the treating physician.

Alternatively, a fixed number of electrodes arranged in a fixed pattern can be implemented. In this implementation, the stimulation parameters (e.g., frequency, amplitude, pattern, duration, etc.) can be tailored or adjusted systematically to determine the initial stimulation settings for closed-loop or open-loop stimulation. Again, this tailoring can be implemented automatically through the algorithm, which incrementally adjusts these characteristics, monitoring the and/or response at each increment until optimal settings are identified. Stimulation therapy can then be applied with these settings, according to the algorithm, which can be dictated by the requirements of the treating physician.

The control unit and the architecture of the system may be designed to constantly optimize stimulation by monitoring the quality of nerve recruitment periodically or on a pulse-by-pulse basis, with the goal of keeping recruitment strength to a minimum (which can reduce muscle twitching) and to minimize the stimulation energy being delivered through the skin.

The EMG recording feature can be capable of detecting both M-wave and F-wave responses, which can be used as feedback inputs (together or independently) to the closed-loop stimulation algorithm to determine the level of activation of the stimulated peripheral nerve. An M-wave response can be an effective indicator of nerve recruitment. F-wave responses also are effective indicators of nerve recruitment, and also indicate that the stimulation-evoked peripheral nerve action potential has activated motor neurons in the associated spinal cord nerves/nerve plexus. For example, an F-wave response to tibial nerve stimulation indicates that the tibial nerve action potential has activated motor neurons in the sacral spinal cord/sacral plexus. F-waves are, however, more difficult to detect. M-wave detection can therefore be better-suited for detecting nerve recruitment in some cases.

The wearable transcutaneous electrical stimulation device can be used to stimulate various peripheral nerves in order to treat medical conditions associated with those nerves. For example, the system can be used to apply electrical stimulation to the tibial nerve to treat pelvic floor dysfunction, e.g., overactive bladder (OAB) medical conditions. As another example, the system can be used to apply electrical stimulation to the tibial nerve to treat sexual dysfunction. In this manner, it is believed that tibial nerve stimulation could be used to treat genital arousal aspects of female sexual interest/arousal disorder by improving pelvic blood flow. In yet another example, the system can be used to apply electrical stimulation to the tibial nerve to treat plantar fasciitis.

To stimulate the tibial nerve, the system can be applied to the ankle area to facilitate transcutaneous tibial nerve stimulation. To facilitate measuring EMG responses to the tibial nerve stimulation, the recording electrodes can be applied to the bottom of the foot, for example, to measure responses from the abductor hallucis and/or flexor hallucis brevis muscles, which are associated with movements of the big toe. The EMG signal can also be used as a control signal to adjust the stimulation parameters and/or stimulation electrode patterns.

As another example, the system can be applied to the wrist area to provide stimulation to the ulnar nerve and/or median nerve. The stimulation electrode array can, for example, be placed on the inside of the lower arm anywhere 0 to 20 cm from the wrist line. EMG recording electrodes can be placed on the base of thumb to record signal from abductor/flexor pollicis brevis. EMG recording electrodes alternatively or additionally can be placed on the base of pinky to record signal from abductor/flexor digiti minimi brevis. The nerve activation could be confirmed by recording M-wave and F-wave EMG signals from the relevant muscles. The EMG signal can also be used as a control signal to adjust the stimulation parameters and/or stimulation electrode patterns. This technology can be applied to median nerve activation for pain management in carpal tunnel syndrome, hypertension management, and nerve conduction study/nerve injury diagnosis for median/ulnar nerve neuropathy, etc.

As a further example, the system can be used to apply transcutaneous electrical stimulation to provide neurostimulation to peripheral nerves in order to enhance nerve regeneration after peripheral nerve injury.

Implementing closed-loop control, the system can utilize measured EMG responses to detect and obtain data related to the electrical activity of muscles in response to the applied stimulation. This data can be used as feedback to tailor the application of the electrical stimulation. Additionally or alternatively, the system can also implement MMG sensors, such as accelerometers, to measure the physical response of the muscles. Other feedback, such as impedance measurements between electrodes and other biopotential recording, can also be utilized. Through this closed-loop implementation, the system can utilize techniques such as current steering and nerve localization to provide peripheral nerve stimulation therapy for treating various medical conditions.

### Improving Electrode Durability

Improved electrode durability can improve the effectiveness of stimulation electrodes in applying transcutaneous electrical neurostimulation. Improved electrode durability can also improve the effectiveness of recording electrodes in measuring M-waves and/or F-waves in response to neurostimulation/neuromodulation.

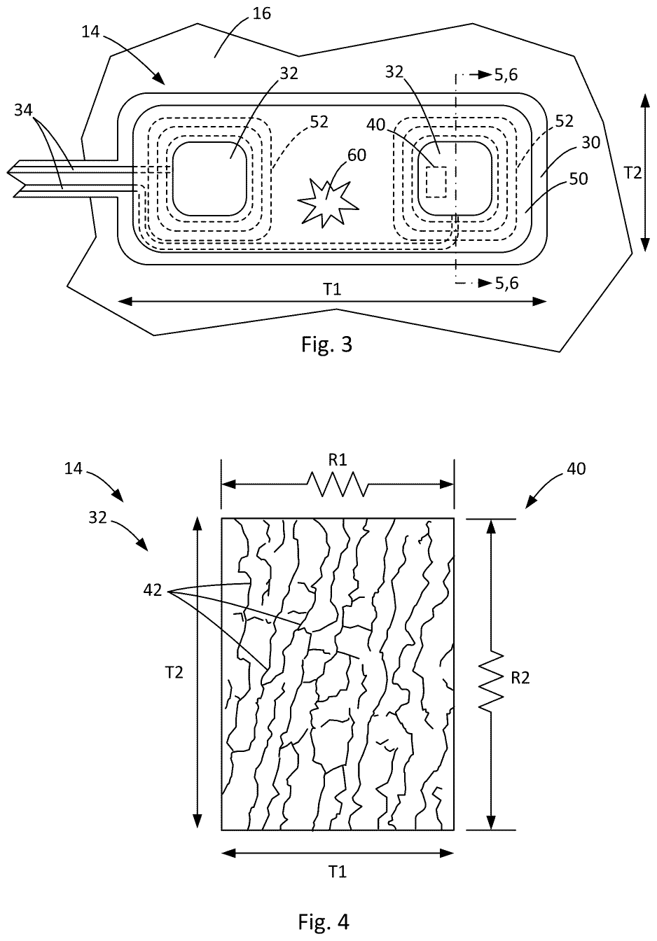

FIGS. 2A and 2B illustrate an example implementation of the neuromodulation system 10 in which the electrodes 14 are integrated with a wearable 16 so that donning the wearable positions the electrodes at the desired location on the subject's anatomy. In the example implementation of FIGS. 3A and 3B, the wearable 16 is an ankle brace that is fixed to the subject via straps 24 (e.g., hook and loop fasteners), with one strap wrapping around the foot and one strap wrapping around the ankle. As shown, the wearable 16 is configured to position electrodes 14 in the area of the ankle and on the bottom of the foot. As shown in FIG. 2B, the wearable 16 can also support the controller 12.

The neuromodulation system 10 described herein is not, however, limited to the illustrated wearable 16 or to the arrangement of the electrodes 14 shown in FIGS. 2A and 2B. This disclosure is directed to the configuration of the garment and electrodes, and is agnostic as to where on the subject the electrodes are positioned. As such, the teachings disclosed herein regarding the configurations of the garment and electrodes can be applied to garments configured to position the electrodes at any location on the subject. Examples of locations on the subject where the garment and electrodes can be configured to position the electrodes include one or more skin surface(s), such as a foot, leg, arm, hand, torso, head, or neck of the subject.

In the example implementation of FIGS. 2A and 2B, the electrodes 14 are surface electrodes with electrically conductive contacts/contact pads configured to engage the skin surface of the subject to apply transcutaneous neurostimulation. The electrodes 14 can be used to deliver stimulation energy to the subject, or to record responses, such as EMG responses, elicited by the applied stimulation energy. For example, using the example implementation of the system 10 illustrated in FIGS. 2A and 2B, the electrodes 14 positioned on the ankle can be used to apply stimulation energy, and the electrodes positioned on the bottom of the foot can be used to record EMG responses to the stimulation energy. In some implementations, a hydrogel pad can be positioned overlying the electrodes 14 so that the contact pads are positioned between the electrodes and the skin surface.

The electrodes 14 can have a variety of constructions. In one example construction, the electrodes 14, i.e., the contact pads and the conductive traces that provide an electrical connection of the contact pads to the controller 20 can be formed directly on the wearable 16. For example, the electrodes 14 can be manufactured via screen printing one or more layers of conductive ink onto a thermoplastic polyurethane (TPU) or similar substrate to form the contact pads and traces. Alternative methods for applying the conductive ink to the substrate can also be implemented. For example, conductive ink can be deposited by screen printing, stencil printing, inkjet printing, gravure, flexographic printing, aerosol jet printing, or combinations thereof.

In some embodiments, the conductive layers can be cured by thermal curing, UV curing, photonic curing, or sintering. In some embodiments, the reinforcing layer is patterned to overlap a boundary of the contact pad by a selected overlap distance to reduce crack formation at the ink edge.

The substrate is then heat transferred to the fabric of the wearable 16. The wearable 16 thus acts as a support for the electrodes 14, which allows the electrodes to deflect with the wearable. As a result, the electrodes 14 can conform perfectly to the contour of the subject's skin, thus producing and maintaining an ideal electrical contact with the skin.

To enable the electrodes 14 to be used with the wearable 16, the electrode is configured to have physical properties offering some degree of compliance so that the electrode can conform, along with the wearable, to the subject's skin. In one example configuration, the flexible TPU substrate allows for the electrode 14 to comply to the wearable 16 and to the subject's skin. In doing so, however, the electrode 14 can be susceptible to bending and/or stretching during use, which can cause the non-flexible conductive layers, i.e., the contact pads, and even the conductive traces, to undergo stress and strain, potentially leading to material failures, such as fissures, cracks, or tears. When the electrode 14 is subjected to cyclic loads of this nature, the conductive material forming the contact pads has a higher propensity to form these material failures along one axis. Due to the discontinuity created by the failures, the conductivity of the contact pads can be reduced, primarily along the axis orthogonal to the failure direction.

For instance, in the example implementation of FIGS. 2A and 2B, the electrodes 14 can be subjected to cyclical forces during walking, running, or other foot/ankle movements. These mechanical forces can begin to wear on the thin layers of conductive ink forming the contact pads of the electrodes 14. Material fatigue can result in material failures of the contact pads on a microscopic level, which can be detrimental to electrical conduction. While detrimental to the delivery of comparatively high amplitude signals associated with electrical stimulation, this can be particularly detrimental in case of low amplitude signals, such as EMG response signals measured by the electrodes 14. In this instance, material failure of the contact pads can thus introduce large measurement errors or a total loss of signal. Damage occurring due to repetitive/cyclical forces is illustrated in FIGS. 3 and 4. FIG. 3 shows a portion of the wearable 16 that includes an electrode 14 according to an example configuration. FIG. 4 illustrates a magnification of a portion 40 of the electrode 14.

In some instances, microcracks/fractures can produce localized high-impedance regions that distort current distribution during stimulation. Such distortion can increase the required compliance voltage for a constant-current stimulation, which increases power consumption and, potentially, perceived discomfort. Distortions in current distribution can also reduce the repeatability and reliability of recruited nerve responses. In sensing applications, microcracks/fractures and resistance drift can increase noise and susceptibility to motion artifacts, which can degrade EMG sensing.

The example electrode configuration of FIG. 3 shows the substrate 30, that is transferred onto the material/fabric of the wearable 16. The electrode 14 is printed or otherwise deposited on the substrate 30, and moves, bends, deflects, etc. along with the portion of the wearable 16 upon which it is applied. In this example configuration, the electrode 14 includes a pair of contact pads 32, each of which has a conductive trace 34 leading thereto. For purposes of simplification, the forces acting on the wearable 16 and, thus, on the electrode 14 are shown as purely tensile forces T1 and T2. The forces are shown as being coincident with length and width dimensions of the electrode 14. In actuality, forces other than tension, such as compression, bending, torsion, etc. can act on the electrode 14 alone or in any combination. Additionally, forces acting on the electrode 14 can do so in any direction.

FIG. 4 shows a magnified area 40 of a contact pad 32. Referring to FIG. 4, tension forces T1 are shown as being greater than tension forces T2. This can occur, for example, as a result of excessive or repetitive stretching or bending of the material of the wearable 16 to which the electrode 14 is secured. As a result of the differential between the elasticity of the substrate 30 and the conductive material 36 forming the contact pad 40, fracture or other material failure can occur, leading to the formation of material failures 42 in the conductive material, which are referred to herein as cracks, but which can be any type of material failure (e.g., fissures, tears, etc.) that creates discontinuity in the conductive material. Because the tension T1 is greater than the tension T2, the cracks 42 extend longitudinally (vertically as shown in FIG. 4). This could be the case, for example, where repeated bending of the electrode 14 produces tension in one direction. For instance, in the example configuration of FIGS. 2A and 2B, the electrode 14 on the bottom of the foot could undergo cyclical bending as the toes/metatarsals flex while the subject is walking.

The cracks 42 produce localized areas of discontinuity, which results in reduced conductivity and increased electrical resistance, both of which effect the resistance of the electrode 14, specifically the contact pads 32. In the example of FIG. 4, with the cracks 42 extend longitudinally, the resulting resistance across the contact pad 32 can be greater laterally as viewed in the figure, i.e., across R1, than vertically, i.e., across R2. across the pad generally vertically as shown in the figure. Because of this, it will be appreciated that the resistance of the electrode 14 can change over time. This can be detrimental to the effectiveness of the neurostimulation system 10 because changes in electrode resistance directly affect the stimulation energy delivered to the subject.

In certain embodiments, the electrode 14 is configured to reduce the rate of resistance increase (or impedance increase) over time and/or to reduce directional anisotropy caused by cracks. For example, maintaining electrical continuity around a periphery of the pad can provide alternative current paths such that regions separated by cracks remain electrically coupled.

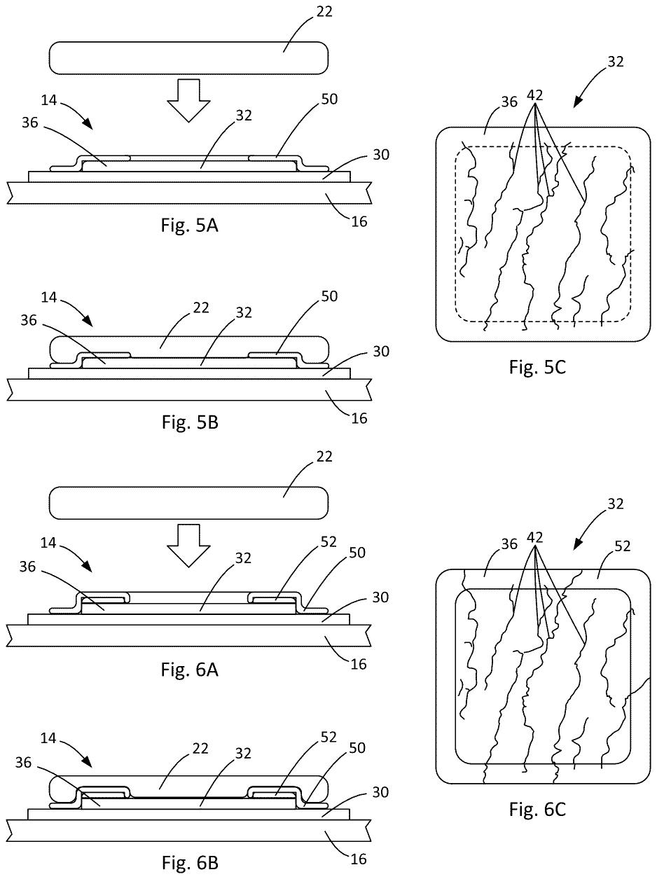

To combat these effects, the electrode 14 is configured to prevent localized bending/stretching/deformation in the areas of the contact pads 32. Example configurations of this are shown in FIGS. 5A-5B and 6A-6B. In these cross-sectional figures, for purposes of illustration, various layers are shown having exaggerated thicknesses. In actuality, the various layers/components are very thin, e.g., on the millimeter or sub-millimeter scale.

Referring to FIG. 5A, a reinforcing layer 50 on the substrate 30 covers a portion of the electrode 14 and substrate 30. The reinforcing layer 50 and encircles the electrode pads 32 and covers peripheral portions 36 (“peripheries”) of the electrode pads 32. While the example configurations of the electrode 14 shown in the figures includes a single reinforcing layer 50 that covers the peripheries 36 of both electrode pads 32, the electrode could include separate, individual reinforcing layers-one associated with each electrode pad.

The reinforcing layer 50 can be configured as a continuous frame or as a plurality of segments spaced around the periphery. A segmented configuration can include concentric rings ring, frames, lattice structure, ribs, or islands and can provide strain-limiting behavior while preserving flex compliance in selected directions. In some embodiments, the reinforcing layer overlaps the contact pad by a predetermined overlap width to reduce stress concentration at the ink edge.

The reinforcing layer 50 increases the effective thickness of the substrate 30, which increases its strength and resistance to deformation in the areas covered by the reinforcing layer. This helps prevent the portions of the substrate 30 and contact pads 32 covered by the reinforcing layer 50 from stretching, bending, deforming, etc. The uncovered portions of the substrate 30, i.e., the uncovered portions of the contact pads 32, while not reinforced, is still at least partially protected due to the coverage of the surrounding portions of the substrate 30. The electrode 14 configuration of FIGS. 5A and 5B provides the requisite contact while, at the same time, limiting the bending, stretching, tension deflection, etc. during use that could lead to cracking that can compromise the electrode conductivity.

The reinforcing layer 50 can be constructed of a thermoplastic polyurethane (TPU) material that is the same of that with which the substrate 30 is constructed. The reinforcing layer 50 could, however, be constructed of a different material. In the example configuration of FIGS. 5A and 5B, the reinforcing layer 50 is separate from the substrate 30, which allows the reinforcing layer to be constructed of any desired material, including TPU. In this configuration, the reinforcing layer 50 could, for example, be applied to the substrate 30 in a separate step, such as via a lamination step or a printing step. As shown in FIGS. 5A and 5B, the hydrogel pad 22 can be applied to the electrode 14 and, due to its gelled consistency, will conform to the surface of the electrode so as to provide effective and reliable conductivity between the electrode pad 32 and the skin surface.

In the example configuration of FIGS. 5A and 5B, the peripheries 36 of the contact pads 32 are sandwiched between the respective TPU layers of the substrate 30 and the reinforcing layer 50. The peripheries 36 of the contact pads 32 are thus reinforced on both sides, which helps prevent the formation of cracks 42 (see FIG. 4) or other failures. Because of this, the reinforcing layer 50 helps ensure that the peripheries 36 of the contact pads 32 maintain a high degree of conductivity by helping to eliminate or reduce the formation of cracks. Advantageously, this improves the conductivity of the contact pads 32 in their entireties. This is represented in the illustration of FIG. 5C.

Referring to FIG. 5C, due to the fact that the reinforcing layer 50 reinforces the periphery 36 of the contact pad 32, the periphery can be kept predominantly free from cracks 42 that can result during use. As shown, some cracks 42 can form in the exposed stimulation area 38 of the contact pad 32, but they can be reduced in number, again, due to the reinforcement of the periphery 36. Some minor cracking can also extend into the periphery 36. Advantageously, however, because the periphery 36 remains largely intact and spans across the ends of the cracks 42 in the exposed portion of the contact pad 32, conductivity is maintained about/across the periphery 36 of the contact pad. As a result, conductivity between any two given points on the contact pad 32, whether covered by the reinforcing layer 50 or not, is maintained, despite any cracks 42 that may form. In other words, any cracks 42 that may form do not act to isolate any portion(s) of the contact pad 32 from the remainder of the contact pad. In this manner, the reinforcing layer 50 imparts durability to the electrode 14 due to its maintaining the conductivity of the contact pads 32 despite the occurrence of some cracking due to repeated use over time.

The reinforcing layer 50 can reduce crack initiation at the ink edge by reducing strain gradients. In some embodiments, the reinforcing layer reduces shear between the ink and the substrate, thereby improving adhesion and reducing delamination. In some embodiments, the reinforcing layer is configured to limit out-of-plane bending in the peripheral region while allowing controlled compliance in the central region.

Another example configuration of the electrode 14 is shown in FIGS. 6A-6C. The configuration of FIGS. 6A-6C is similar to the configuration of FIGS. 5A-5C, with differences that are described herein below. In the example configuration of FIGS. 6A-6B, it is the configuration of the reinforcing layer 50 that differs from that of the example configuration of FIGS. 5A-5B. In the example configuration of FIGS. 6A-6B, the substrate 30 and contact pad 32 do not differ from the configuration of FIGS. 5A-5B, nor does the configuration and application of the hydrogel pad 22.

According to the example configuration of FIGS. 6A-6B, the reinforcing layer 50 can include a conductive layer 52 configured to extend along and overlie the periphery 36 of the contact pad 32. The conductive material used to form the conductive layer 52 can be the same material used to form the contact pads 32 or a different material. Examples include gold, silver, copper, stainless steel, or combinations/alloys thereof. In one example configuration, the conductive layer 52 can be constructed of a metal that is softer/more ductile than that which forms the contact pads 32. In this manner, the conductive layer 52 can be less susceptible to crack formation.

Referring to FIG. 6C, due to the conductive, crack-resistant nature of the conductive layer 52, electrical conductivity is maintained along the periphery 36 of the contact pad 32. As shown, some cracks 42 can form in the exposed stimulation area 38 of the contact pad 32, but they can be reduced in number due to the reinforcement of the periphery 36 by the reinforcing layer 50. Advantageously, even if some cracking forms in the periphery 36, the conductive layer 52 remains largely intact and maintains electrical conductivity along the entire periphery. As a result, conductivity between any two given points on the contact pad 32 is maintained despite any cracks 42 that may form. In this manner, in addition to the reinforcing layer 50, the conductive layer 52 imparts durability to the electrode 14 due to its maintaining the conductivity of the contact pads 32 despite the occurrence of some cracking due to repeated use over time.

In certain embodiments, the conductive layer 52 can be electrically coupled to the contact pad 32 at multiple points around the periphery to provide redundant conductive paths. In some embodiments, the conductive layer 52 is insulated from direct skin contact by the reinforcing layer 50 and/or an encapsulant layer 60, thereby maintaining patient safety and controlling the effective stimulation interface.

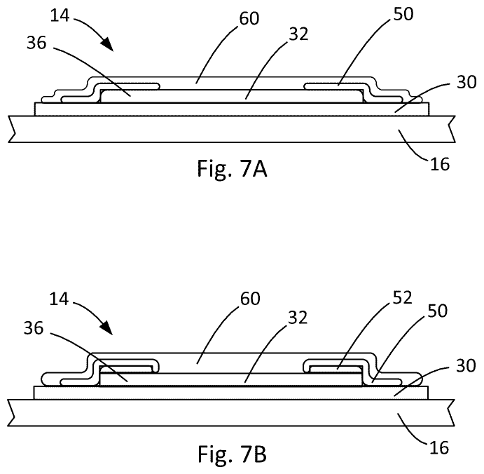

According to an other example configuration, in addition to the reinforcing layer 50 and/or the conductive layer 52, the electrode 14 can include a thin top layer of encapsulant ink 60. This is shown generally in FIG. 3 and in greater detail in FIGS. 7A and 7B. FIG. 7A illustrates the encapsulant ink layer 60 implemented on an electrode 14 having the configuration of FIGS. 5A-5C. FIG. 7B illustrates the encapsulant ink layer 60 implemented on an electrode 14 having the configuration of FIGS. 6A-6C.

In FIGS. 7A and 7B, the encapsulant ink layer 60 is illustrated as being applied across a majority of the upper surface of the electrode 14. The encapsulant ink layer 60 could, however, be applied only where necessary, e.g., on the surface of the contact pad 32. The encapsulant ink forming the layer 60 is generally capacitive in nature and allows alternating current to flow unimpeded while offering a protective layer on top of the surface of the contact pad 32 where contact is made with the subject's skin. The encapsulant ink layer 60 can be applied in multiple configurations, including a full layer covering the electrode, a perforate layer that allows direct current transmission via perforations, or a combination thereof where the encapsulant area partially covers portions the contact pad 32, while leaving other portions of the contact pad exposed.

In some embodiments, perforations can be configured as an array of openings having a selected density and distribution to control current density and/or to provide a consistent effective electrode area. In some embodiments, the encapsulant layer can be configured to reduce sweat/moisture ingress and to maintain stable electrode impedance during prolonged wear.

Electrode durability can be evaluated by subjecting an electrode to repeated bending and/or stretching cycles representative of use (e.g., flexion at a joint or repeated bending at the metatarsals). In comparative examples, electrodes without a reinforcing layer can exhibit increased resistance and/or impedance and reduced EMG sensing fidelity after repeated cycles, whereas electrodes including a reinforcing layer as described herein can maintain electrical continuity between regions of the contact pad and exhibit reduced drift. The terms “within a predetermined range” and “substantially constant” can refer to ranges selected based on a desired therapy or sensing specification, such as maintaining impedance suitable for delivering a commanded constant current within a stimulator compliance voltage.

From the above, it will be appreciated that the electrode configurations described herein can facilitate a method, which can include placing a wearable carrying one or more reinforced electrodes as described above on a subject, delivering stimulation, recording EMG responses, and adjusting stimulation parameters and/or electrode selection based on the recorded responses. Improved durability can reduce recalibration frequency and improve session-to-session repeatability.

Additionally, while the system and apparatus disclosed herein are described in terms of their respective structures and configurations, it will be appreciated that this description also discloses the method by which the system and apparatus, especially the electrode, are manufactured.

From the above description of the invention, those skilled in the art will perceive improvements, changes and modifications. Such improvements, changes and modifications within the skill of the art are intended to be covered by the appended claims.

Having described the invention, the following is claimed:

1. An electrode for applying transcutaneous electrical stimulation, comprising:
a flexible substrate;
an electrically conductive contact pad printed on the substrate with conductive ink; and
a reinforcing layer applied onto the substrate and covering a periphery of the contact pad, the reinforcing layer being configured to reinforce the periphery of the contact pad to inhibit material failures of the conductive ink along the periphery;
wherein the reinforcing layer is configured to maintain electrical continuity along the periphery of the contact pad in order to provide electrical continuity across portions of the contact pad within the periphery that are left exposed by the reinforcing layer.

2. The electrode recited in claim 1, wherein the flexible substrate comprises a first polymer sheet onto which the contact pad is printed, and the reinforcing layer comprises a second polymer sheet laminated onto the flexible substrate.

3. The electrode recited in claim 2, wherein the first and second polymer sheets comprise thermoplastic polyurethane (TPU) sheets.

4. The electrode recited in claim 1, wherein the reinforcing layer comprises a layer of conductive material configured to overlie the periphery of the contact pad.

5. The electrode recited in claim 4, wherein the layer of conductive material is configured to contact the periphery of the contact pad to provide electrical conductivity across material failures of the contact pad.

6. The electrode recited in claim 1, further comprising an encapsulant ink layer that covers the contact pad.

7. The electrode recited in claim 6, wherein the encapsulant ink layer fully covers contact pad.

8. The electrode recited in claim 6, wherein the encapsulant ink layer comprises a perforate layer that allows direct current transmission via perforations that expose portions of the contact pad.

9. The electrode recited in claim 6, wherein the encapsulant ink layer partially covers contact pad and leaves exposed portions of the contact pad configured for skin contact.

10. The electrode recited in claim 1, further comprising a hydrogel pad configured to overlie the contact pad.

11. An apparatus for applying transcutaneous electrical stimulation, comprising:
a wearable structure configured to be worn by a subject;
at least one electrode according to claim 1 mounted on the wearable; and
a controller configured to control the operation of the electrode to apply electrical stimulation energy to the subject.

12. An apparatus as recited in claim 11, wherein the at least one electrode comprises an electrode array comprising a plurality of electrodes, wherein the controller is configured to selectively activate subsets of electrodes for stimulation and/or recording to perform current steering and/or nerve localization.

13. A neurostimulation system comprising the apparatus of claim 12, wherein the controller is configured to deliver stimulation via at least one stimulation electrode of the plurality of electrodes and to record electromyogram (EMG) responses via at least one recording electrode of the plurality of electrodes,
wherein the reinforced periphery of the contact pads of the at least one stimulation electrode maintains electrical continuity along the periphery of the contact pad in order to provide electrical continuity across portions of the contact pad within the periphery so that stimulation can be applied evenly across the contact pad despite the formation of discontinuities in the contact pad, and
wherein the reinforced periphery of the contact pads of the at least one recording electrode maintains electrical continuity along the periphery of the contact pad in order to provide electrical continuity across portions of the contact pad within the periphery so that the EMG responses can be recorded with reduced drift despite the formation of discontinuities in the contact pad.

14. A method of manufacturing an electrode for transcutaneous electrical stimulation, comprising:
printing an electrically conductive contact pad on a flexible polymer substrate using a conductive ink;
applying a reinforcing layer over a peripheral region of the contact pad while leaving a central region exposed; and
coupling the electrode to a wearable structure;
wherein the reinforcing layer reduces strain in the peripheral region during cyclic deformation to inhibit crack formation in the conductive ink.

15. The method of claim 14, wherein applying the reinforcing layer comprises laminating a polymer film onto the substrate.

16. The method of claim 14, further comprising applying a conductive peripheral layer that overlies and electrically couples to the peripheral region to provide redundant conductive paths across cracks.

17. An electrode for transcutaneous electrical stimulation, comprising: a flexible substrate; a printed conductive contact pad; and a reinforcing structure coupled to a peripheral region of the contact pad; wherein the electrode is configured such that an electrical resistance and/or impedance of the contact pad remains within a predetermined range after repeated bending and/or stretching cycles representative of wear.

18. The electrode of claim 17, wherein the reinforcing structure comprises a peripheral frame that remains electrically conductive around the contact pad to maintain electrical continuity between discontinuous regions of the contact pad.

19. The electrode of claim 17, wherein directional anisotropy of resistance across the contact pad is reduced relative to an otherwise identical electrode lacking the reinforcing structure.
